(19) United States
(12) Patent Application Publication
(10) Pub. No.: US 2005/0267492 A1
(43) Pub. Date: Dec. 1, 2005

(54) Surgical instrument for specimen retrieval

(76) Inventors: Philippe Poncet, Sandy Hook, CT (US); Scott Carpenter, Fremont, CA (US)

Correspondence Address: CANTOR COLBURN, LLP, 55 GRIFFIN ROAD SOUTH, BLOOMFIELD, CT, 06002, US

(21) Appl. No.: 10/844,018

(22) Filed: May 12, 2004

(51) Int. Cl. A61B 17/24

(52) U.S. Cl. 606/114

(57) ABSTRACT

Disclosed herein is a collection device for manipulating matter in an inaccessible space, comprising a clamp in mechanical communication with an actuator means, wherein the clamp has an inflexion point; a barrier membrane in mechanical communication with the clamp, wherein the expansion of the barrier membrane is controlled by the clamp. Disclosed herein is a collection device for manipulating matter in an inaccessible space, comprising a clamp in mechanical communication with an actuator means, wherein the clamp comprises at least two elements that can control a barrier membrane and wherein the at least two elements are not in mechanical communication with one another; a barrier membrane in mechanical communication with the at least two elements of the clamp, wherein the expansion of the barrier membrane is controlled by the clamp.

### BACKGROUND

This disclosure relates to surgical instruments with specimen retrieval capabilities that can be utilized during the course of arthroscopic, laparoscopic and/or endoscopic and/or endoluminal procedures.

Laparoscopic, endoscopic and endoluminal surgical procedures are minimally invasive procedures in which operations are carried out within the body by means of elongated instruments inserted through small entrance openings (via ports, cannulas or percutaneously) in the body. The entrance opening in the body to allow passage of the endoscopic or laparoscopic instruments to the interior of the body may be a natural passageway of the body, or it can be created by a tissue-piercing instrument, such as a trocar or needle. Some laparoscopic and endoscopic procedures require that any instrument or instrumentation inserted in the body be sealed, i.e., provisions must be made to ensure that gases do not enter or exit the body through the instrument or the entrance incision so that the surgical region of the body, may be insufflated (pneumoperitoneum). Actuation of such instruments is generally constrained to the movement of the various components along a longitudinal axis with means provided to convert longitudinal movement to lateral movement. Because the endoscopic or laparoscopic cannulas, instrumentation, and any desired punctures or incisions are relatively narrow, endoscopic or laparoscopic surgery is less invasive and causes much less trauma to the patient as compared with traditional procedures in which the surgeon is required to—create large incisions in body tissue.

Minimally invasive procedures are often used to operate on and partially or totally remove body tissue or organs from the interior of the body, e.g. nephrectomy, cholecystectomy, appendectomy, oopherectomy, and other such laparoscopic procedures. During such procedures, it is common that a tissue sample, cyst, tumor or other diseased or inflamed tissue or organ, such as the gallbladder for example, may be removed via the access opening in the skin, or through a cannula. Various types of entrapment devices are used to facilitate this procedure. Such retrieval procedures are difficult when operating in the confined space of the body cavity or through a small arthroscopic or other endoscopic incision or body aperture.

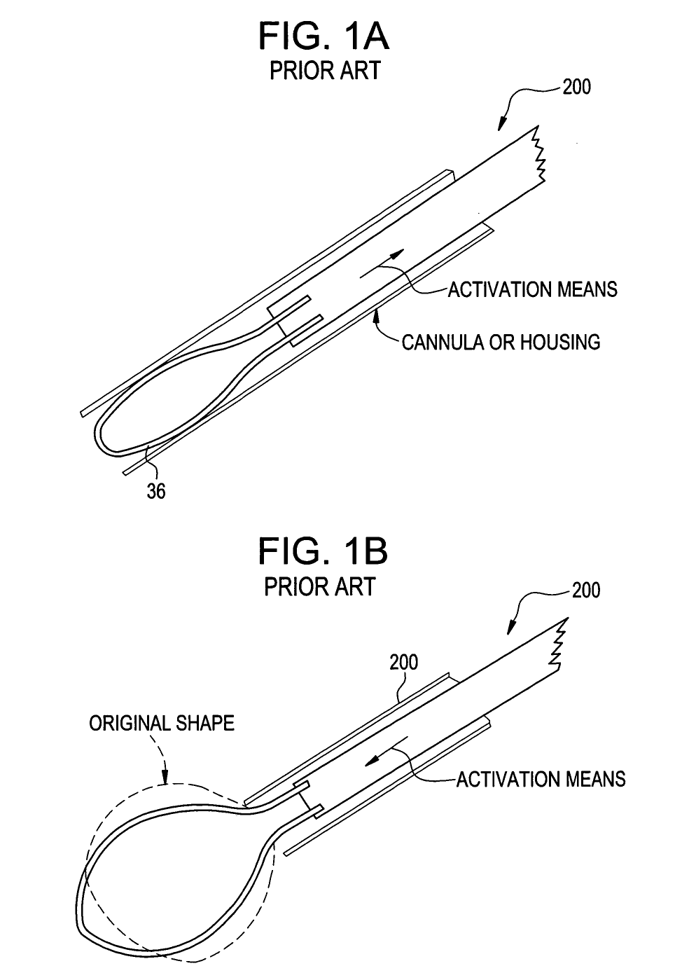

Many forms of apparatus for performing such surgical operations have been previously proposed using flexible steel elements, which spring apart when extended from the distal end of a tube and which can be brought together again on withdrawal back into the tube. However, these devices are not completely satisfactory for various reasons. One of these reasons is indicated in the FIG. 1 below, which depicts a collection device 200 comprising a clamp 36 contained in a cannula or housing. The clamp 36 is predeformed to have a mouth whose original shape is circular (as shown by the dotted lines). However, after storage in the housing as shown in FIG. 1(a), the clamp 36 undergoes plastic deformation. This plastic deformation results in a clamp that has a shape that is no longer circular upon deployment, but rather is oval in shape as may be seen in the FIG. 1(b). Such plastic deformation may result in a clamp that is not reusable or does not open sufficiently any longer. In order to overcome such plastic deformation, and restore the clamp to its original shape, the clamp has to be reworked mechanically and, potentially, thermally. Such repeated mechanical and thermal cycling of the clamp will eventually lead to failure of the material of the clamp. The use of such a clamp in the collection device is cost prohibitive.

It is therefore desirable to provide an apparatus for performing surgical operations, which can be repeatedly used and for which interchangeable components may be utilized, so as to minimize costs.

### BRIEF SUMMARY

Disclosed herein is a collection device for manipulating matter in an inaccessible space, comprising a clamp in mechanical communication with an actuator means, wherein the clamp has an inflexion point; a barrier membrane in mechanical communication with the clamp, wherein the expansion of the barrier membrane is controlled by the clamp.

Disclosed herein is a collection device for manipulating matter in an inaccessible space, comprising a clamp in mechanical communication with an actuator means, wherein the clamp comprises at least two elements that can control a barrier membrane and wherein the at least two elements are not in mechanical communication with one another; a barrier membrane in mechanical communication with the at least two elements of the clamp, wherein the expansion of the barrier membrane is controlled by the clamp.

Disclosed herein is a surgical apparatus for manipulating matter at an intended manipulation temperature in a confined or inaccessible space, comprising a housing; a clamp in located at the distal end of the housing in slideable communication with the housing, wherein the clamp has an inflexion point; a barrier member in communication with the clamp; and an actuating means located at the proximal end of the housing, for extending the clamp from the housing to manipulate matter within the space and for withdrawing the clamp into the housing, the arrangement being such that the clamp bends or twists in a lateral or helical sense to manipulate the matter on extending from the housing at the manipulation temperature, and wherein the clamp becomes relatively straightened on withdrawal into the housing at the manipulating temperature.

Disclosed herein is a method for manipulating matter within a confined space or an inaccessible space inside a living being comprising inserting into the body of a living being a surgical apparatus comprising a housing; a clamp in located at the distal end of the housing in slideable communication with the housing, wherein the clamp has an inflexion point; a barrier member in communication with the clamp; and an actuating means located at the proximal end of the housing and in mechanical communication with the clamp; extending the clamp from the housing to manipulate matter within the space; and withdrawing the clamp into the housing.

Disclosed herein is a method for manipulating matter within a confined space or an inaccessible space inside a living being comprising inserting into the body of a living being a surgical apparatus comprising a housing; a barrier member in communication with the clamp; a clamp in mechanical communication with an actuator means, wherein the clamp comprises at least two elements that can control a barrier membrane and wherein the at least two elements are not in mechanical communication with one another; and further wherein the clamp is located at the distal end of the housing in slideable communication with the housing; and an actuating means located at the proximal end of the housing; extending the clamp from the housing to manipulate matter within the space; and withdrawing the clamp into the housing.

### DETAILED DESCRIPTION OF DRAWINGS

FIG. 1 is a schematic depiction of problems with the prior art;

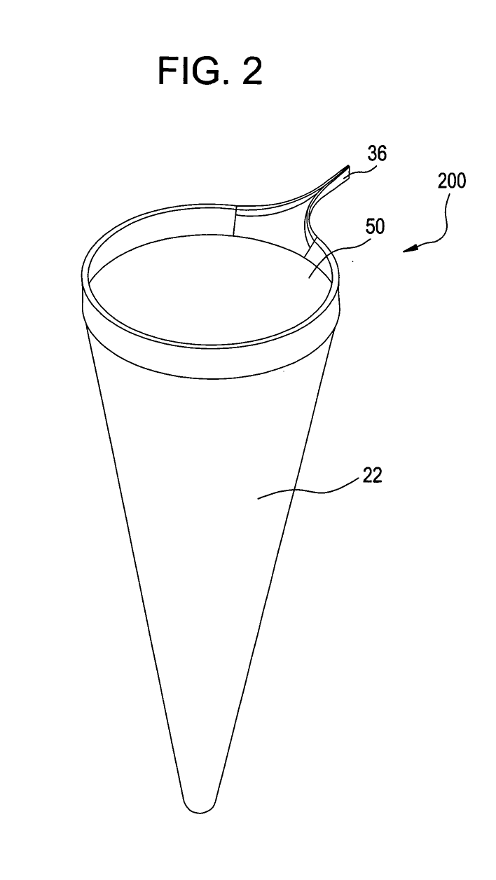

FIG. 2 depicts one exemplary embodiment of the device, which comprises a clamp and a barrier membrane;

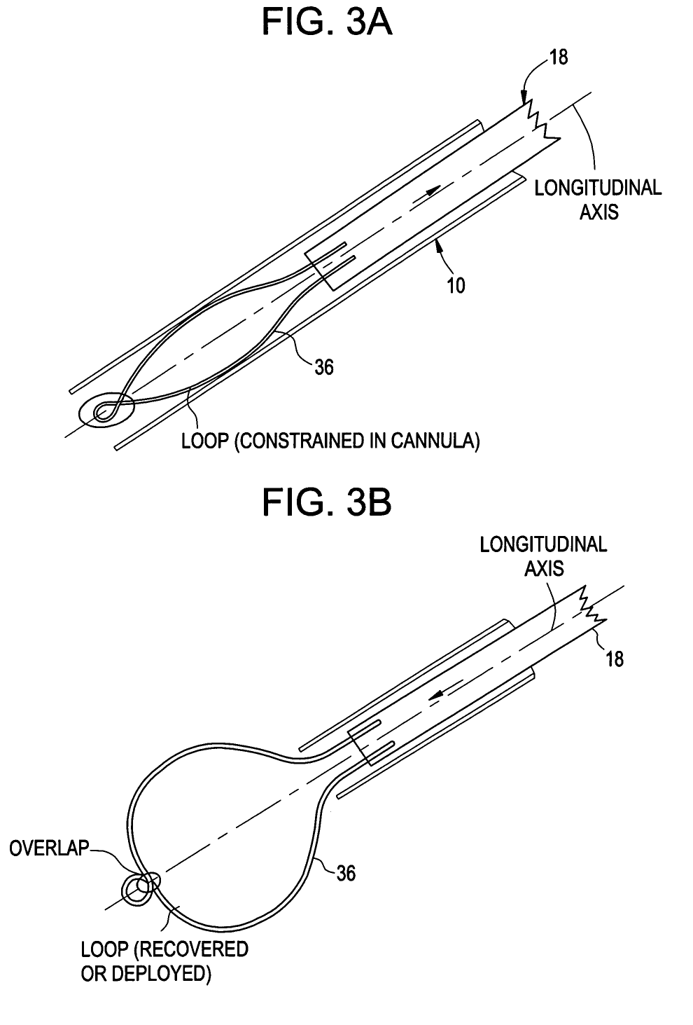

FIG. 3 is a schematic depicting one exemplary embodiment of a clamp contained in a housing, wherein the clamp is designed to have a shape that minimizes any plastic deformation at the inflexion point and allows spring back to original shape;

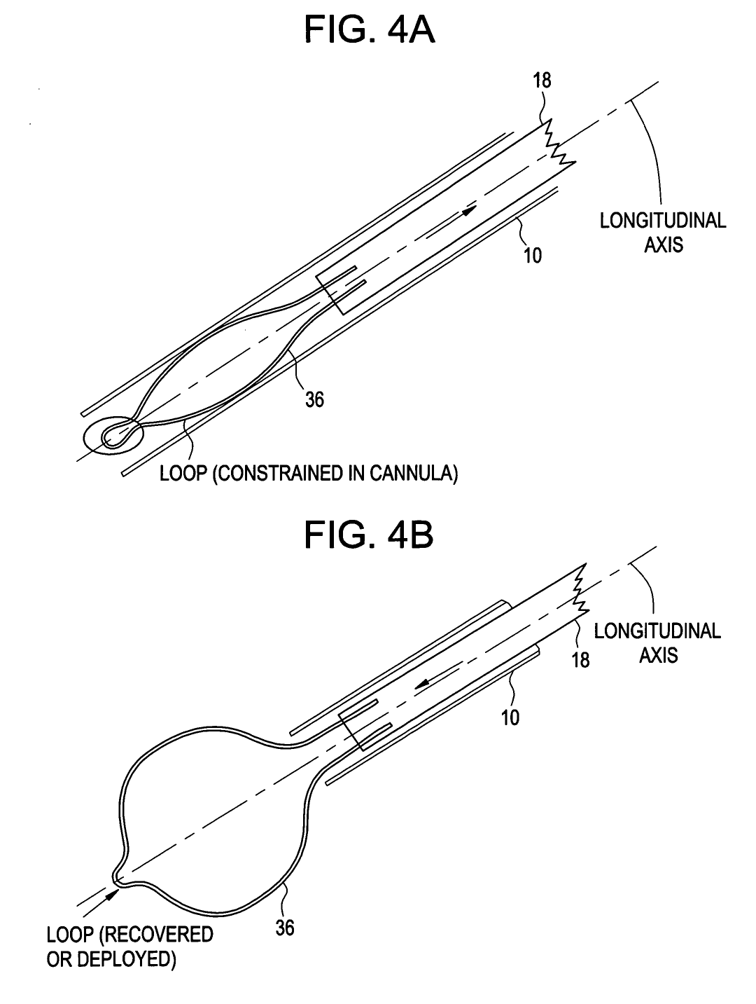

FIG. 4 is another schematic depicting one exemplary embodiment of a clamp contained in a housing, wherein the clamp is designed to have a shape that minimizes any plastic deformation at the inflexion point and allows spring back to original shape;

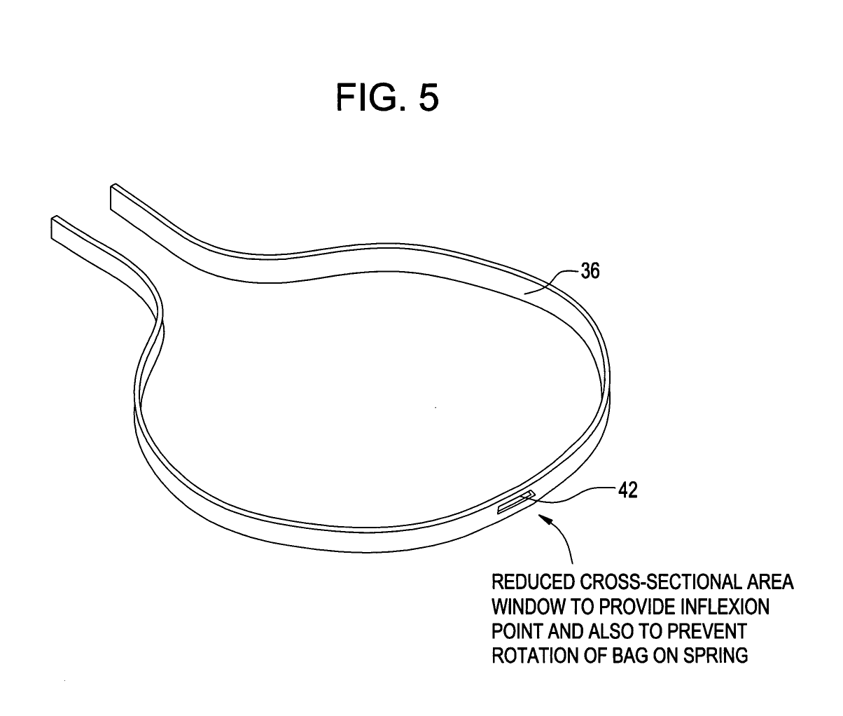

FIG. 5 is a depiction of one exemplary embodiment of the clamp, where the inflexion point is formed by the reduced cross-sectional area;

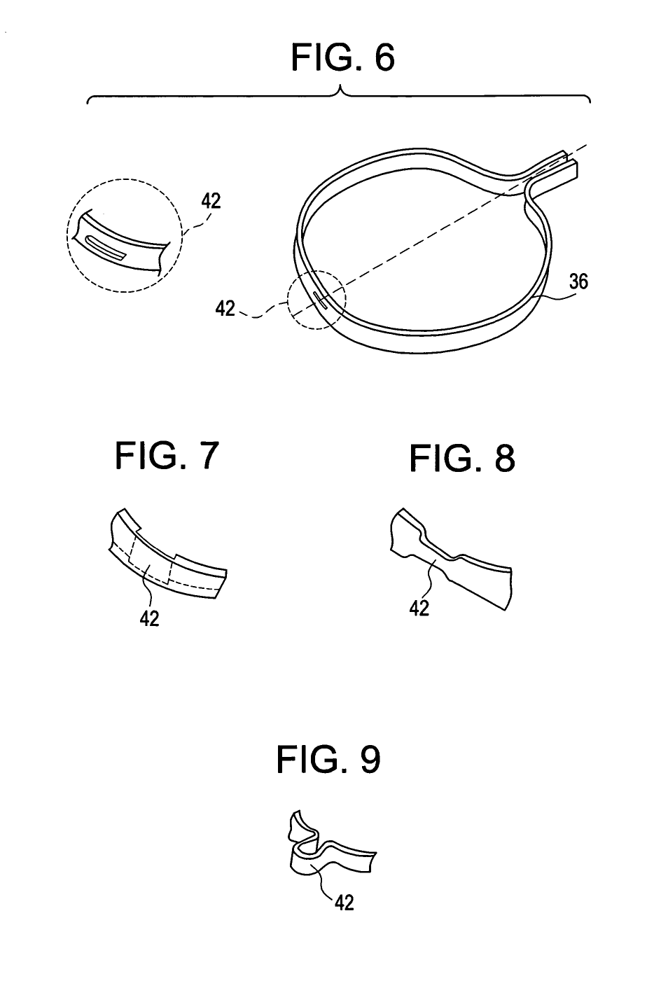

FIG. 6 is a schematic showing one exemplary embodiment of the inflexion point on the clamp, where the inflexion point is formed by the reduced cross-sectional area;

FIG. 7 is a schematic showing one exemplary embodiment of the inflexion point on the clamp, where the inflexion point is formed by the reduced cross-sectional area;

FIG. 8 is a schematic showing one exemplary embodiment of the inflexion point on the clamp, where the inflexion point is formed by the reduced cross-sectional area;

FIG. 9 is a schematic showing one exemplary embodiment of the inflexion point on the clamp, where the inflexion point is formed by the use of a predeformed shape;

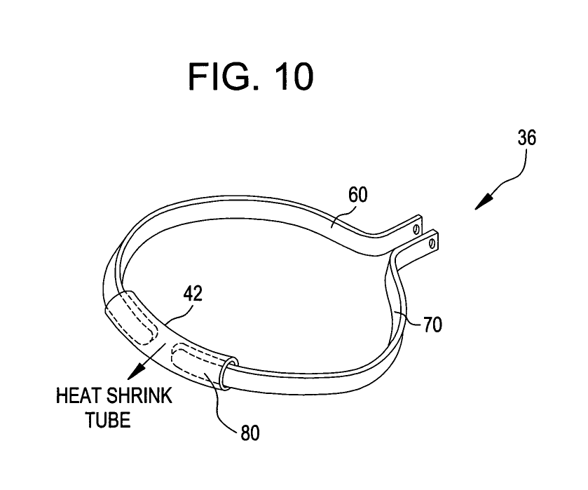

FIG. 10 is a depiction of another exemplary embodiment of the clamp wherein the clamp is comprises three elements, wherein the third element is manufactured from a material having a lower elastic modulus than the material from which the first two elements are constructed and performs as a ‘hinge’;

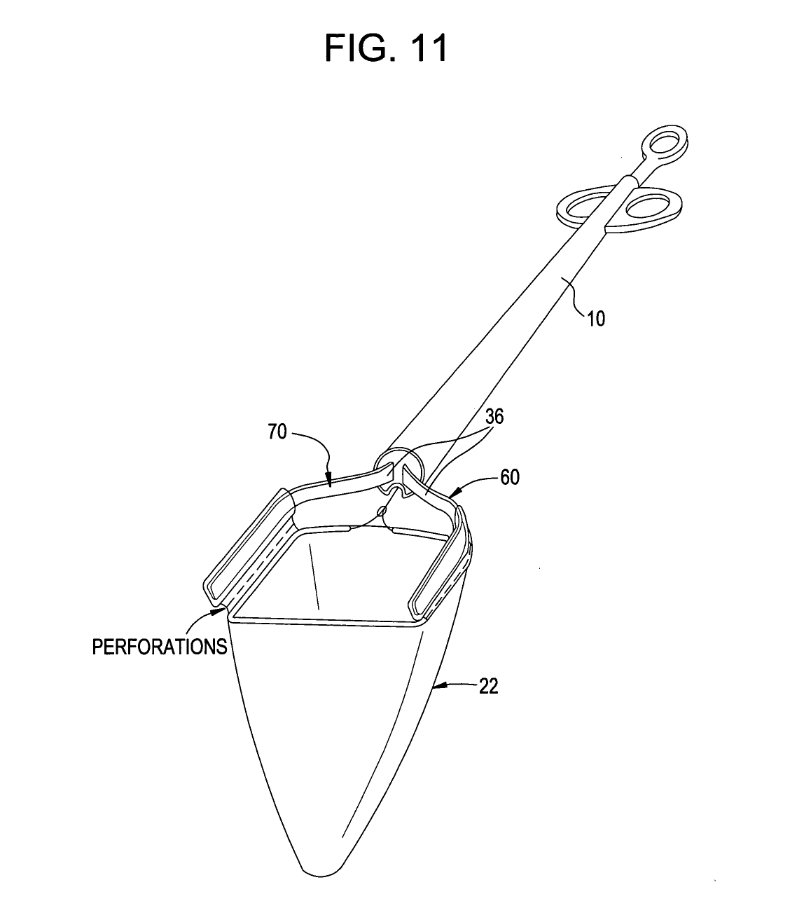

FIG. 11 depicts one exemplary embodiment wherein the clamp comprises two elements that are not in physical communication with each other;

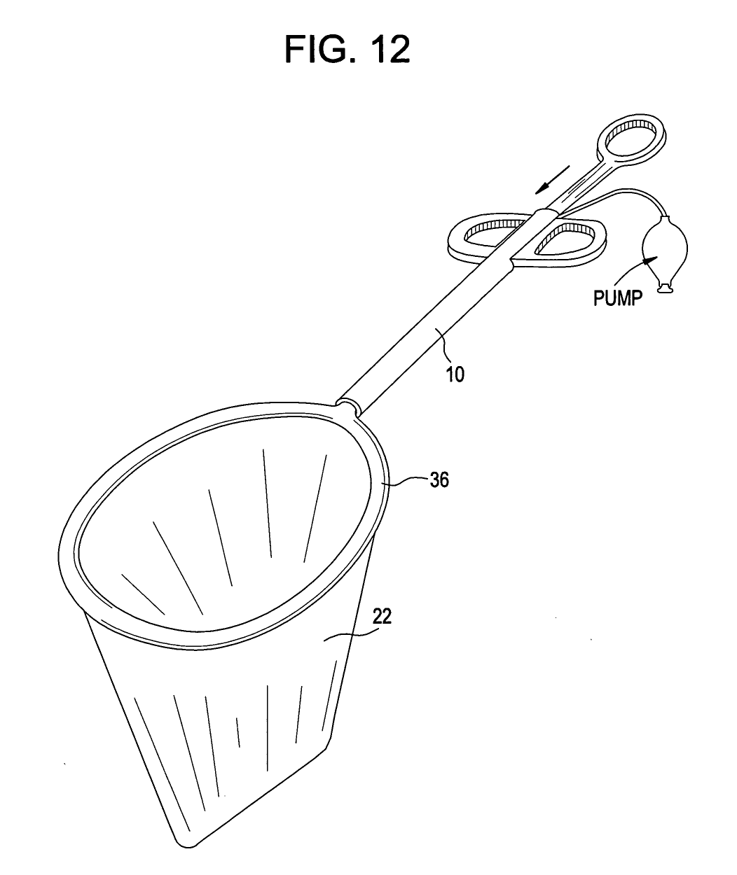

FIG. 12 depicts one exemplary embodiment wherein the clamp is manufactured from an expandable conduit, wherein the clamp is in the form of a loop;

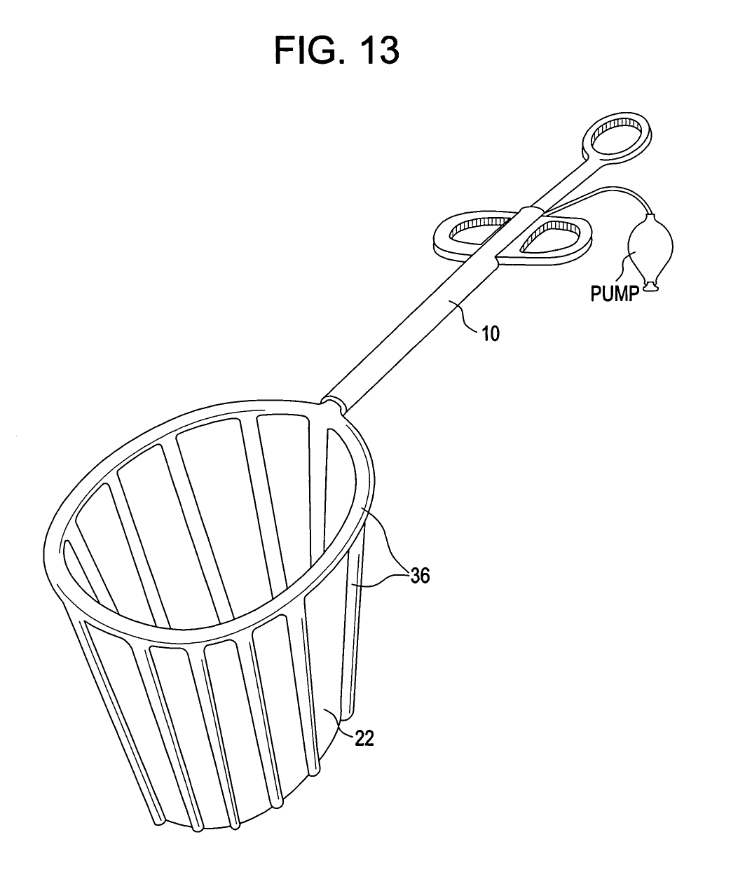

FIG. 13 depicts another exemplary embodiment wherein the clamp is manufactured from an expandable conduit, and wherein the clamp is in the form of a web to enhance 3-dimensional deployment of the barrier;

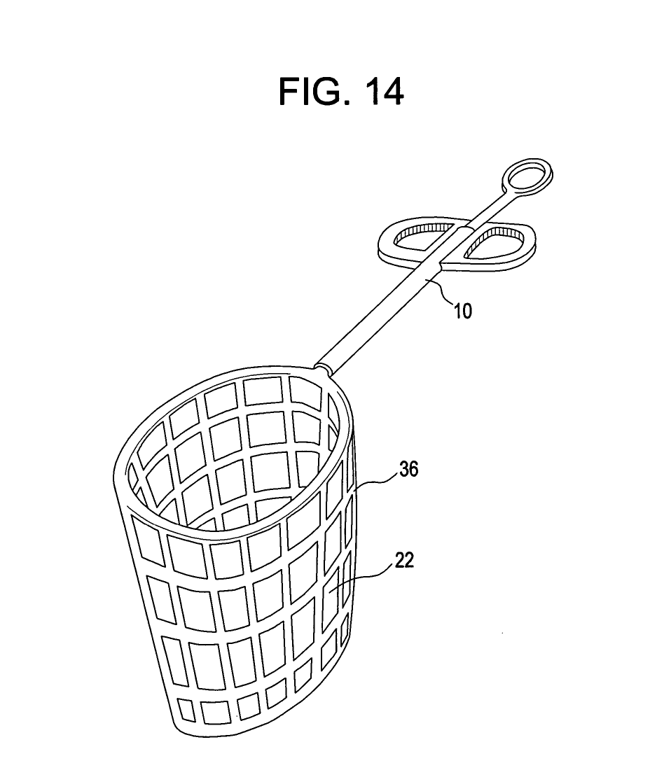

FIG. 14 depicts another exemplary embodiment wherein the clamp is manufactured from a conduit, and wherein the clamp is in the form of a grid to enhance 3-dimensional deployment of the barrier;

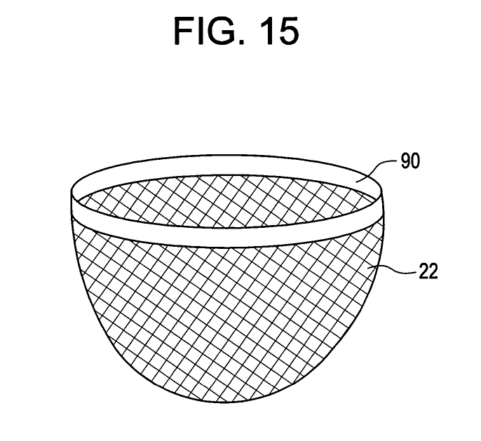

FIG. 15 is a schematic depicting one exemplary embodiment of a barrier membrane embedding a spring made from an elastically deformable material to enhance 3-dimensional deployment of the barrier;

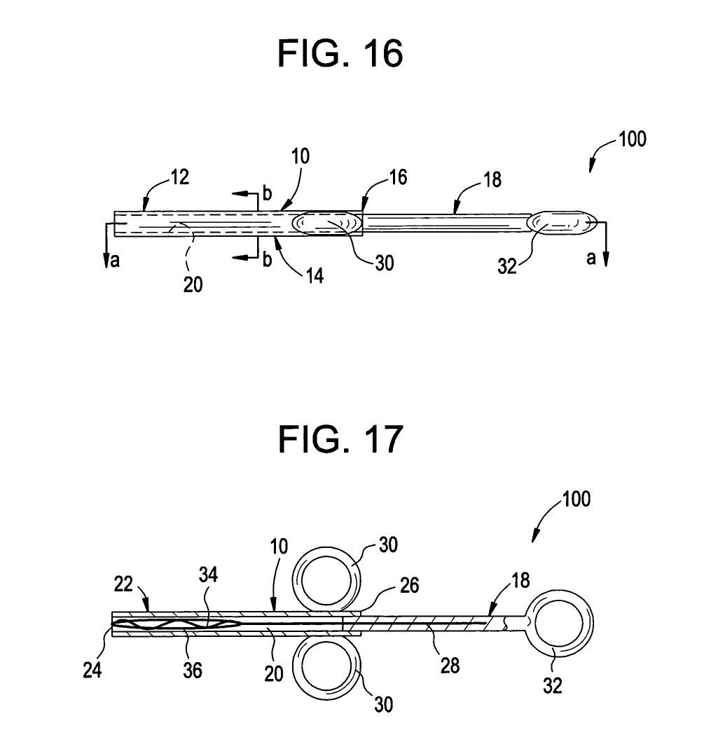

FIG. 16 is a schematic depicting one exemplary embodiment of a deployment device shown ready for insertion thru a port, trocar or cannula;

FIG. 17 is a schematic depicting one exemplary embodiment of a deployment device;

FIG. 18 is a schematic depicting one exemplary embodiment of the use of a deployment device in which barrier has been deployed by actuator (once in the body), including capture of a specimen;

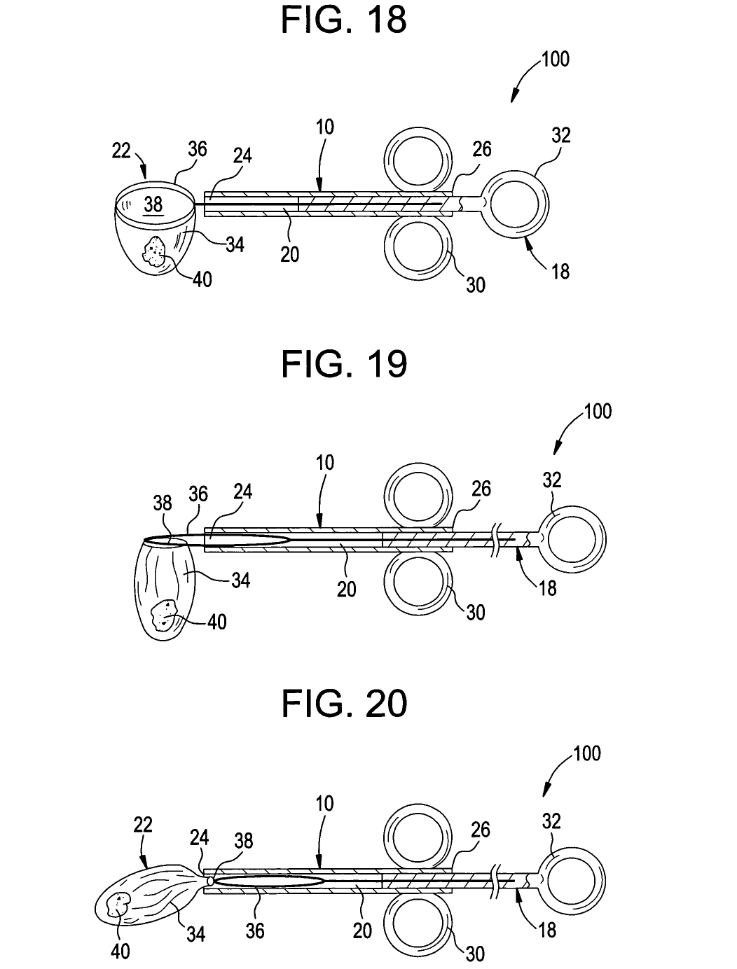

FIG. 19 is a schematic depicting one exemplary embodiment of the use of a deployment device and capture of a specimen inside the barrier/pouch shown with the clamp has been separated from the pouch; and

FIG. 20 is a schematic depicting one exemplary embodiment of the use of a deployment device with the pouch containing the specimen being cinched/enclosed.

### DETAILED DESCRIPTION OF PREFERRED EMBODIMENTS

Disclosed herein is a collection/retrieval device for manipulating matter in a confined or inaccessible space, comprising a clamp and a barrier membrane. The clamp is in mechanical communication with an actuating means and may be manufactured from a metal, a polymer, or a combination of a metal and a polymer. The actuating means generally permits the clamp to expand or contract thereby respectively opening or closing a mouth to the barrier membrane. The barrier membrane is advantageously manufactured from a polymer. The clamp and the barrier membrane are in mechanical communication with one another in a manner such that either the expansion or the contraction of the clamp may be used to respectively open or close the mouth to the barrier membrane. In one embodiment, the expansion of the clamp facilitates the opening of the mouth of the barrier membrane. In another embodiment, the contraction of the clamp facilitates the contraction of the barrier membrane. The collection device, which can be reusable, is advantageously protected in a hollow housing, within which it can be sterilized and preserved in its initial state when not in use. The hollow housing also comprises an actuating means for extending the collection device from the housing to manipulate matter within the space and for withdrawing the collection device into the housing. In one embodiment, the barrier membrane can be cinched, detached and left behind in the body during a surgical procedure. It can then be retrieved at the end of the procedure through one of the port incision in the skin. When such a procedure is utilized during a surgery, the housing is only utilized to collapse and withdraw the clamp.

It is to be noted that as used herein, the terms “first,” “second,” and the like do not denote any order or importance, but rather are used to distinguish one element from another, and the terms “the”, “a” and “an” do not denote a limitation of quantity, but rather denote the presence of at least one of the referenced item. Furthermore, all ranges disclosed herein are inclusive of the endpoints and independently combinable.

The collection device may be advantageously utilized for manipulating matter in the interior of living beings such as, for example, a human being or an animal during a laparoscopic, endoluminal or endoscopic surgery. The collection device may also be advantageously used for removing or retrieving sample tissue from the interior of the patient for a biopsy or an autopsy. The device also advantageously permits multiple specimen retrieval from a single patient without the need for multiple devices. When utilized for multiple specimen retrieval, it can be advantageously used to isolate a first retrieved specimen from a second retrieved specimen. As used herein, the terms laparoscopic, endoscopic, arthroscopic, endoluminal are interchangeable and refer to instruments having a relatively narrow operating portion for insertion into a cannula, a port, a trocar, a sheath or a small incision in the skin, or to a surgical procedure in which such instruments are employed. Use herein of any one of the foregoing terms (i.e., laparoscopic, endoscopic, arthroscopic, endoluminal, or the like) should not be construed in such a manner as to exclude the other terms.

With reference now to an exemplary embodiment displayed in FIG. 2, the collection device 200 comprises a clamp 36 and a barrier membrane 22. The barrier membrane 22 is in mechanical communication with the clamp 36. In one embodiment, the barrier membrane 22 is permanently attached to the clamp 36. In another embodiment, the barrier membrane 22 is temporarily attached to the clamp 36, such that it can be removed (detached) and cinched after it has been utilized. The detached barrier membrane 22 may be discarded or it may be sterilized and reutilized if so desired. When detached, a new barrier membrane 22 may be attached to the clamp 36, so that the entire system can be reused. The clamp 36 is constructed in such a manner so that the barrier membrane 22 may be detached when desired.

In one embodiment, the clamp 36 has a shape and a perimeter length such that it can repeatedly mechanically communicate with the barrier membrane 22 during a surgery. The clamp 36 is reusable and it permits insertion, support and opening of the mouth of the barrier membrane 22 inside an inaccessible space located in a patient's interior. The clamp 36 can contain connecting means (not shown), such as, for example, slots, hooks (e.g., Velcro), magnets, threads, clips, adhesive coatings, or the like, for attaching and supporting the barrier membrane 22 during storage and during surgical procedures, when the collection device 200 is being used.

In one embodiment, the clamp 36 can be made from a single element that is deformed to have a mouth 50 with a circular shape, a semi-circular shape, an elliptical shape, a square shape, a rectangular shape, a triangular shape, a polygonal shape, or a combination comprising at least one of the foregoing shapes. In another embodiment, the clamp 36 can be made from two or more elements that do not physically contact one another but can be activated to form the mouth 50 that has one of the foregoing shapes. In yet another embodiment, the clamp 36 can be made from two or more elements that physically contact one another and can be activated to form the mouth 50 that has one of the foregoing shapes. The mouth 50 formed by opening the clamp 36 may not be in a single plane and may not be in the same plane as the longitudinal axis of the device.

The elements used to form the clamp 36 can be in the form of a ribbon, a wire comprising a single or multiple filaments, a braided wire, a tube, or the like, or a combination comprising at least one of the foregoing forms. The elements used in the clamp 36 can have a cross sectional area that is rectangular, square, circular, triangular, hollow (e.g., a conduit), or the like, or a combination comprising at least one of the foregoing cross-sectional areas. When the clamp 36 is made from a single element it is desirable for the clamp 36 to have a point of inflexion, which absorbs most of the energy when the clamp 36 is deflected as it is constrained in the housing, delivery system or deployment tube. This energy is released when the clamp 36 is removed from the housing during deployment. The release of this energy promotes the clamp 36 to open a mouth 50 (not shown) in the barrier membrane 22.

As noted above, the clamp 36 is contained in a housing 10 when it is not being deployed during a surgery. FIGS. 3 and 4 depict exemplary embodiments of the housing 10 containing the clamp 36 prior to actuation and after deployment. The clamp 36 can be repeatedly deformed from its initial shape to a variety of different shapes to facilitate the use of the barrier membrane 22 (not shown) during a surgery. As may be seen from the FIGS. 3 and 4 the clamp 36 can be slideably reciprocated into the housing 10 or out of it by the actuator means 18. From FIGS. 3 and 4 it may be seen that the clamp 36 is designed to have an inflexion point 42 that can absorb and release energy upon repeated deformation and whose presence permits the clamp to repeatedly return to its designed shape in the body cavity or operating space.

The inflexion point 42 is that point on the clamp 36, which is subjected to maximum deflection/strain upon deforming/constraining the clamp 36. The presence of the inflexion point on the clamp 36 can permit the clamp to display spring like behavior. The clamp 36 can return to its original shape when a force is applied to the clamp via the actuating means to return it to the housing. There may be more than one inflexion point 42 on the clamp 36 if desired and as a function of the particular design. In one embodiment, the inflexion point 42 is a section of the clamp 36 that has a lower elastic modulus and/or a lower cross-sectional area than the other portions of the clamp 36, so that it can be easily deformed upon the application of a force via the actuation means. While the inflexion point 42 may be positioned at any point on the clamp 36, it is generally desirable for the inflexion point 42 to be positioned on the clamp 36 at a point at which the circumference of the clamp 36 intersects with the longitudinal axis of the clamp 36, when the longitudinal axis of the clamp 36 is superimposed upon the longitudinal axis of the housing 10. In one embodiment, the longitudinal axis of the clamp 36 is any axis that divides the clamp into equal parts. In another embodiment, the longitudinal axis of the clamp 36 is that axis that divides the clamp into equal halves. The longitudinal axis of the housing is the longitudinal axis of the tube forming the housing. The longitudinal axis of the tube is the axis that passes through the geometric center of each of the ends of the tube utilized for the housing.

The clamp 36 may be constructed from a single element or multiple elements. In one exemplary design, when the clamp 36 is constructed from a single element, it is generally desirable to have a section on the clamp 36 containing an inflexion point. It is to be noted that clamps 36 comprising multiple elements can also contain inflexion points. FIG. 5 is a depiction of one exemplary embodiment of the clamp 36 made from a single element. In FIG. 5, the clamp 36 is made from a ribbon of either a metal or a polymer and has a rectangular cross-sectional area. The inflexion point is created by a reduction in the cross sectional area. Various exemplary designs embodying a reduced surface area to create an inflexion point 42 in the clamp 36 may be seen in the FIGS. 6, 7, and 8. In FIGS. 6, 7, and 8, the cross-sectional area of the clamp 36 is reduced by the formation of a notch or a hole. The notch may be V-shaped, U-shaped, W-shaped, or the like, or a combination comprising at least one of the foregoing shapes. The hole may have the shape of a circle, a slot, a diamond, and ellipse, or the like, or a combination comprising at least one of the foregoing shapes. In one embodiment, the notch may be located at the edge of the clamp 36. In another embodiment, as in the FIGS. 5 and 6, the hole may be located in the middle of the clamp 36 such that it does not contact the edges of the clamp 36. The reduced cross sectional area forms the inflexion point in the clamp 36 such that upon the application of a force, a deflection or deformation of the clamp 36 occurs primarily at the inflexion point, and in some cases prevents plastic deformation at the inflection point.

In another embodiment, the clamp 36 has a predeformed section that forms the inflexion point. The predeformed section is created by pre-deforming a section of the clamp in a manner such that the bend includes an internal angle of less than or equal to about 180 degrees as measured by the angle between tangents taken from two points on the bend when these tangents meet on the longitudinal axis that divides the clamp into two equal halves when the clamp is in its undeformed state. In one embodiment, the predeformed section is created by pre-deforming a section of the clamp in a manner such that the bend includes an internal angle of less than or equal to about 90 degrees as measured by the angle between tangents taken from two points on the bend when these tangents meet on the longitudinal axis that divides the clamp into two equal halves when the clamp is in its undeformed state. In yet another embodiment, the predeformed section is created by pre-deforming a section of the clamp in a manner such that the bend includes an internal angle of less than or equal to about 60 degrees as measured by the angle between tangents taken from two points on the bend when these tangents meet on the longitudinal axis that divides the clamp into two equal halves when the clamp is in its undeformed state. The tangents to the bend are taken at the two points of maximum curvature, one on either side of the longitudinal axis that divides the clamp into two equal halves, i.e., that point at which the ratio of the change in the radius of the bend to the change in angle (as measured from the longitudinal axis that divides the clamp 36 into two equal halves) is maximum.

It is to be noted that when the inflexion point contains a predeformed section that does not have curved surfaces but rather is created by straight edges, then the internal angle created by the extension of the straight edges at their point of intersection with the longitudinal axis can be less than 90 degrees, preferably less than 60 degrees.

In an exemplary embodiment depicted in the FIGS. 3, 4 and 9, the clamp 36 has a predeformed section, which facilitates the deformation of the clamp 36 when a force is applied to it via the actuating means and the connecting means. While FIG. 9, depicts a predeformed U-shaped section, other geometries, such as, for example, a predeformed V-shaped section, a predeformed W-shaped section, a predeformed M-shaped section, a predeformed S-shaped section, a predeformed O-shaped section (similar to that depicted in the FIG. 3), or the like, or a combination comprising at least one of the following predeformed sections may be used. The inflexion point 42 may be created by combining a reduced cross-sectional area with a predeformed section, if desired.

In one embodiment, the inflexion point in the clamp 36 may be produced by using a second material having a different elastic modulus at that region of the clamp, where the inflexion point 42 is desired. The second material behaves as a hinge and facilitates in the creation of the inflexion point 42. The second material generally has a different composition from the composition of the first material forming the remainder of the clamp 36. It is generally desirable for the second material located at the inflexion point 42 to have a lower elastic modulus from the first material that forms the remainder of the clamp 42. FIG. 10 is a depiction of another exemplary embodiment of the clamp 36 wherein the clamp is comprises of three elements. The first two elements 60 and 70 are manufactured from a first material, while the third element 80 is manufactured from a second material that has a lower elastic modulus than the metal used in the manufacture of the first two elements 60 and 70 respectively. The two elements made from the first material are in physical communication with the second material via a slot in the second material as can be seen in the FIG. 10. The elements manufactured from the first material may be in physical communication with the elements manufactured from the second material in either a temporary or a permanent manner. If the physical communication is temporary, then the elements manufactured from the second material may be replaced when desired.

Other modes of physical communication between the first and the second elements may be advantageously employed, such as, for example, the use of crimping, heat shrinkage, an adhesive or glue, a spot weld, a tongue and groove joint, a dovetail joint, a spline joint, a dowel joint, a rabbet joint, a lap joint, a mitre joint, a mortise and tenon joint, a box joint, or the like, or a combination comprising at least one of the foregoing joints. In one embodiment, the first material can be a metal, while the second material can be a polymer. In another embodiment, the first material can be a shape memory alloy, while the second material can be a polymer. When a polymer is used as a second material, it may be employed as the element 80 in the form of a heat shrink tube. In order to vary the properties of the inflexion point (i.e., its ability to deform under stress), it may be desirable to reduce the cross-sectional area of the second material if desired.

In another embodiment, the clamp 36 may comprise two elements that are not in physical contact with each other as shown in FIG. 11. The two elements 60 and 70 are in mechanical communication with the barrier membrane 22, which can be attached or detached as desired. The two elements form a U-shaped clamp 36 which can be used to support the barrier membrane 22. The barrier membrane 22 may be maintained in position on the clamp 36 by means of slots contained in the barrier membrane 22. The barrier membrane 22 contains perforations by means of which it can be easily removed from the clamp 36.

As noted above, the clamp 36 can also be manufactured from a conduit. Suitable cross-sections for the conduit are circular, square, rectangular, triangular, polygonal, or the like, or a combination comprising at least one of the foregoing cross-sections. When the clamp 36 has a cross section in the form of a conduit, it is generally desirable for the clamp to be made from a material that is easily deformable, such as, for example, a polymeric elastomer, so that it can expand and contract upon the application of an external stimulus. This external stimulus may be heat, electricity or pressure. Heat can be applied through resistive heating. Pressure may be applied by using a fluid. The pressure may be applied by a pneumatic and/or hydraulic actuator means. One exemplary means of the application of pressure is via a hand pump as shown in the FIGS. 12 and 13. Another exemplary means of the application of pressure is via a syringe. In yet another exemplary means, the application and removal of pressure may be accomplished by an appropriate control system having artificial intelligence. Application of pressure promotes an expansion of the clamp 36 due to inflation, while the contraction of the clamp 36 is due to deflation that occurs upon the removal of the applied pressure.

In FIG. 12, the clamp 36 comprises an inflatable loop while in FIG. 13 the clamp 36 comprises an inflatable loop with an attached inflatable web. The clamp 36 may also comprise an inflatable loop with an attached inflatable grid as shown in FIG. 14. In these embodiments, the deformable clamp 36 may be expanded by using compressed gas or saline solution delivered to the clamp 36 by a pump located at an actuator opening of the housing. The pump constitutes the actuator means. The barrier membrane 22 is in mechanical communication with the clamp 36 and may be permanently attached or temporarily attached if desired. In one embodiment, the barrier membrane 22 and the clamp 36 can be manufactured from a single piece of material.

In the use of the device depicted in the FIGS. 13 and 14, either the barrier membrane 22 and/or clamp 36 may be deployed simultaneously or sequentially. The term “deployed” as used herein refers to the inflation of the loop and/or the web or grid. If the web or grid is inflated prior to the loop, then it is assumed that the barrier membrane 22 has been deployed first, while if the loop is inflated prior to the web or grid, then it is assumed that the clamp 36 has been deployed first. When the clamp 36 and the barrier membrane 22 are deployed sequentially, any particular order may be used. In one exemplary embodiment, it is desirable to first deploy the barrier membrane 22 followed by an inflation of the clamp 36. When the desired object from the interior of the patient is acquired into the barrier membrane 22, the clamp 36 may be deflated thereby trapping the object. The deflated clamp with the barrier membrane 22 may then be withdrawn into the housing 10, following which the housing is removed from the body of the patient.

The barrier membrane 22 is disposable and may have a variety of different geometries for collecting specimens from the interior of the patient. The barrier membrane can have a conical shape or a cylindrical shape, with a first end that it open and in mechanical communication with the clamp 36. The second end of the barrier membrane 22 is sealed off so as to facilitate the capture or collection of desired objects from with the interior of the patient. In one embodiment, the barrier membrane 22 may have the shape of a bag or a pouch. The barrier membrane is preferably made of a flexible and impermeable biocompatible material. The barrier membrane 22 is sufficiently thin that it can be folded or gathered, together with the clamp, to fit within the inside of the housing.

In one preferred embodiment, the barrier membrane material is substantially impermeable to body fluids and other liquids, such as normal saline solution, which might be present during surgical procedures. The thickness of the membrane is sufficient to provide an effective barrier to noxious or contaminated materials such as bile, spillage from inflamed or infected tissues, or tumor cells. In an alternate embodiment, the barrier membrane material is substantially impermeable to tissue samples, but is generally permeable to body fluids and other liquids, such as normal saline solution, which might be present during surgical procedures. In this embodiment, the barrier membrane material can be a knit, net, web, mesh or grid. Suitable materials include perforated, webbed or netted polyethylene, polyvinyl chloride, urethane, polysiloxanes (e.g., silicone rubber), and the like. A similar construct can be made of, or contain, shape memory materials such as shape memory alloys and/or shape memory polymers.

The barrier membrane 22 is manufactured from a polymer, a list of which are given below. The polymer may contain filler particles that are made from ceramics and/or metals. Examples of suitable materials for the barrier membranes 22 are polyurethane membranes, polysiloxane membranes, fluoropolymers membranes such as polytetrafluoroethylene membranes, polyester membranes, polyamide membranes, polyethylene membranes, or the like, or a combination comprising at least one of the foregoing materials. The barrier membrane 22 can be made from textiles (i.e., woven polymers) if desired. The barrier membrane 22 can alternatively be made from blow molded, dip molded or vacuum formed polymers if desired.

It is generally desirable for the barrier membrane 22 to have a tensile strength of greater than or equal to about 400 kilograms/square centimeter (kg/cm²) (6000 pounds per square inch). In one embodiment, the barrier membrane 22 has a tensile strength of greater than or equal to about 450 kilograms/square centimeter (kg/cm²). In another embodiment, the barrier membrane 22 has a tensile strength of greater than or equal to about 500 kilograms/square centimeter (kg/cm²). In yet another embodiment, the barrier membrane 22 has a tensile strength of greater than or equal to about 500 kilograms/square centimeter (kg/cm²).

The barrier membrane 22 preferably has a mounting means for easy mounting to the clamp 36. The mounting means can comprise channels, slots, clips, hooks, springs, or the like, or a combination comprising at least one of the foregoing mounting means for attaching the barrier member 22 to the clamp 36. In one embodiment, the barrier membrane may be made by welding i.e. layers of polymeric films can be welded to the clamp 36 together to form a pouch, which serves as the barrier membrane. The barrier membrane 22 is chemically, ultrasonically or heat welded onto the clamp 36.

In another embodiment, the barrier membrane 22 contains a spring 90 at the open end, which is in mechanical communication with the clamp 36. The spring 90 is made from an elastically deformable material and can be attached to the clamp 36 by using a hook, a magnet, screw-threads, or the like. The spring may be manufactured from an elastically deformable material such as a shape memory alloy or a polymer. If the spring is manufactured from a shape memory alloy, it may be coated with a polymer if desired. The polymeric coating may be used to adhesively or chemically bond the barrier membrane 22 to the clamp 36 if desired. In one embodiment, the spring may have a diameter that is larger than the diameter of the clamp 36 when both the spring and the clamp are in the expanded state. The larger diameter facilitates the support of the barrier membrane by the clamp 36. In one embodiment, the barrier membrane 22 is provided with a locking mechanism at the first end (i.e., mouth) to secure any retrieved organs. The locking mechanism may be a drawstring with a slip-knot to secure the mouth of the barrier membrane.

The clamp 36 may be made from a metal or from a polymeric material. Examples of suitable metals are stainless steel alloys, titanium alloys, cobalt-chrome alloys, nickel-titanium alloys, or the like, or a combination comprising at least one of the foregoing metals. In one embodiment, the metals are shape memory alloys. Alloys such as nickel-titanium that undergo a martensitic transformation may exhibit a “shape memory effect”. As a result of this transformation, the high temperature phase known as “austenite” changes its crystalline structure through a diffusion-less shear process adopting a less symmetrical structure called ‘martensite’. This process may be reversible as in shape memory alloys and therefore upon heating, the reverse transformation occurs. Upon cooling, the starting temperature of the martensitic transformation is generally referred to as the Mₛ temperature and the finishing temperature is referred to as the M_{f} temperature. The starting and finishing temperatures of the reverse or austenitic transformation upon heating are referred to as Aₛ and A_{f} respectively.

Aₜ temperatures above A_{f}, alloys undergoing a reversible martensitic phase transformation may be mechanically deformed and the martensitic transformation can be stress-induced. These alloys generally recover their original shapes upon removal of the mechanical stress. At temperatures above the A_{f}, the stress-induced martensite is not stable and will revert back to austenite upon the release of deformation. The strain recovery associated with the reversion of stress-induced martensite back to austenite is generally referred to as “pseudoelasticity” or “superelasticity” as defined in ASTM F2005, Standard Terminology for Nickel-Titanium Shape Memory Alloys. The two terms are used interchangeably to describe the ability of shape memory alloys to elastically recover large deformations without a significant amount of plasticity due to the mechanically induced crystalline phase change.

When shape memory alloys are used in the clamp 36, it is generally desirable to use an alloy having an austenite transformation finish temperature (A_{f}) below the body temperature. Such an alloy exhibits pseudoelasticity or superelasticity at the body temperature. It is also desirable to use certain shape memory alloys such as nickel-titanium in the cold-worked condition that exhibit linear superelasticity. Such shape memory alloys that exhibit superelasticity or linear superelasticity are especially preferred. The elastic materials herein exhibit greater than 2% recoverable strain. Preferably, the elastic materials herein exhibit greater than 4% recoverable strain.

Suitable shape memory alloys that may be used in the clamp 36 are nickel titanium alloys. Suitable examples of nickel titanium alloys are binary nickel-titanium, nickel-titanium-chromium, nickel-titanium-molybdenum, nickel-titanium-vanadium, nickel-titanium-niobium, nickel-titanium-copper, nickel-titanium-iron, nickel-titanium-hafnium, nickel-titanium-palladium, nickel-titanium-gold, nickel-titanium-platinum alloys, or the like, or combinations comprising at least one of the foregoing nickel titanium alloys. Preferred alloys are binary nickel-titanium alloys.

Nickel-titanium alloys that may be used in the clamp 36 generally comprise nickel in an amount of about 54.5 weight percent (wt %) to about 57.0 wt %, based on the total composition of the alloy. Within this range it is generally desirable to use an amount of nickel greater than or equal to about 54.8 wt %, preferably greater than or equal to about 55.5 wt %, based on the total composition of the alloy. Also desirable within this range is an amount of nickel less than or equal to about 56.5, and more preferably less than or equal to about 56.0 wt %, based on the total composition of the alloy.

An exemplary composition of a nickel-titanium alloy having an Aₛ greater than or equal to about 0° C. is one which comprises about 55.5 wt % nickel (hereinafter Ti-55.5 wt %-Ni alloy) based on the total composition of the alloy. The Ti-55.5 wt %-Ni alloy has an As temperature in the fully annealed state of about 30° C. After cold fabrication and shape-setting heat treatment, the Ti-55.5 wt %-Ni alloy has an Aₛ of about 10 to about 15° C. and an austenite transformation finish temperature (A_{f}) of about 30 to about 35° C.

Another exemplary composition of a nickel-titanium alloy, which comprises about 55.8 wt % nickel (hereinafter Ti-55.8 wt %-Ni alloy) based on the total composition of the alloy. The Ti-55.8 wt %-Ni alloy generally has an Aₛ of −15° C. in its fully annealed state, and an A_{f} of about 0° C. After cold fabrication and shape-setting heat treatment, the Ti-55.8 wt %-Ni alloy generally has an As of about 0° C. and an austenite transformation finish temperature (A_{f}) of greater than or equal to about 20° C.

Another useful alloy that may be used to form the clamp 36 is a β titanium alloy. Suitable β titanium alloys are those wherein the stability of the β phase can be expressed as the sum of the weighted averages of the elements that comprise the alloy, often known as the molybdenum equivalent (MO_{eq.}). P. Bania, Beta Titanium Alloys in the 1990's, TMS, Warrendale, 1993, defines the MO_{eq.} in the following equation (1) as
MO_{eq.}=1.00Mo+0.28Nb+0.22Ta+0.67V+1.43Co+1.60Cr+0.77Cu+2.90Fe+1.54Mn+1.1Ni+0.44W−1.000Al (1)
wherein Mo is molybdenum, Nb is niobium, Ta is tantalum, V is vanadium, Co is cobalt, Cr is chromium, Cu is copper, Fe is iron, Mn is manganese, Ni is nickel, W is tungsten and Al is aluminum and wherein the respective chemical symbols represent the amounts of the respective elements in weight percent based on the total weight of the alloy. It is to be noted that aluminum can be substituted by gallium, carbon, germanium or boron.

Hf (hafnium), Sn (tin) and Zr (zirconium) may also be used in the β titanium alloy and exhibit similarly weak effects on the β stability. Although they act to lower the β transus, these elements are considered neutral additions. US Air Force Technical Report AFML-TR-75-41 has suggested that Zr has a small Mo equivalent of 0.25 while Al is an a stabilizer having a reverse effect to that of Mo. Hence, the Mo equivalent in weight percent is calculated according to the following equation (2), which is a modified form of the equation (1):
MO_{eq.}=1.00Mo+0.28Nb+0.22Ta+0.67V+1.43Co+1.60Cr+0.77Cu+2.90Fe+1.54Mn+1.11Ni+0.44W+0.25(Sn+Zr+Hf)−1.00Al (2).

In general it is desirable to use a shape memory alloy that displays superelasticity and/or pseudoelasticity, which has a molybdenum equivalent of about 7 to about 11 wt %, based upon the total weight of the alloy. In one embodiment, it is desirable to have a shape memory alloy that displays superelasticity and/or pseudoelasticity, which has a molybdenum equivalent of about 7.5 to about 10.5 wt %, based upon the total weight of the alloy. In another embodiment, it is desirable to have a shape memory alloy that displays superelasticity and/or pseudoelasticity, which has a molybdenum equivalent of about 8 to about 10 wt %, based upon the total weight of the alloy. In yet another embodiment, it is desirable to have a shape memory alloy that displays superelasticity and/or pseudoelasticity, which has a molybdenum equivalent of about 8.5 to about 9.8 wt %, based upon the total weight of the alloy.

In one embodiment, in the equations (1) and (2) above, all of the elements may be optional if desired. In another embodiment, the elements that may be present in the composition in addition to titanium are molybdenum, vanadium, chromium, aluminum, and/or niobium. In another embodiment, it is generally desirable for the elements represented in equations (2) to be present in the composition in amounts of greater than or equal to about 0.1, preferably greater than or equal to about 0.5, preferably greater than or equal to about 1, preferably greater than or equal to about 1.5, preferably greater than or equal to about 5, and preferably greater than or equal to about 10 wt %, based upon the total weight of the alloy composition. In yet another embodiment, it is generally desirable for the elements represented in equation (2) to be present in the composition in amounts of less than or equal to about 50, preferably less than or equal to about 40, preferably less than or equal to about 30, preferably less than or equal to about 28, preferably less than or equal to about 25, and preferably less than or equal to about 23 wt %, based upon the total weight of the alloy composition.

A suitable example of a β titanium alloy is one which comprises an amount of about 8 to about 12 wt % of molybdenum, about 2.8 to about 6 wt % aluminum, up to about 2 wt % vanadium, up to about 4 wt % niobium, with the balance being titanium. All weight percents are based on the total weight of the alloy. Within the aforementioned range for molybdenum, it is generally desirable to have an amount of greater than or equal to about 8.5, preferably greater than or equal to about 9.0, and more preferably greater than or equal to about 9.2 wt % molybdenum. Also desirable within this range is an amount of less than or equal to about 11.9, preferably less than or equal to about 11.8, preferably less than or equal to about 11.75, preferably less than or equal to about 11.65, and more preferably less than or equal to about 11.5 wt % molybdenum, based on the total weight of the alloy.

Within the aforementioned range for aluminum, it is generally desirable to have an amount of greater than or equal to about 2.85, preferably greater than or equal to about 2.9, and more preferably greater than or equal to about 2.93 wt % aluminum. Also desirable within this range is an amount of less than or equal to about 5.0, preferably less than or equal to about 4.5, and more preferably less than or equal to about 4.0 wt % aluminum, based on the total weight of the alloy.

Within the aforementioned range for niobium, it is generally desirable to have an amount of greater than or equal to about 2, preferably greater than or equal to about 3, and more preferably greater than or equal to about 3.5 wt % niobium, based on the total weight of the alloy.

In one exemplary embodiment, it is generally desirable for the β titanium alloy to comprise 8.9 wt % molybdenum, 3.03 wt % aluminum, 1.95 wt % vanadium, 3.86 wt % niobium, with the balance being titanium. In another exemplary embodiment, it is generally desirable for the β titanium alloy to comprise 9.34 wt % molybdenum, 3.01 wt % aluminum, 1.95 wt % vanadium, 3.79 wt % niobium, with the balance being titanium.

The metals used in the clamp 36 may be coated with a polymer if desired. The polymer may be a thermoplastic polymer, a thermosetting polymer, or a combination comprising at least one of the foregoing polymers. The polymer may be an oligomer, a copolymer such as a block copolymer, a graft copolymer, a star block copolymer, an ionomer, a dendrimer, a blend of polymers, or a combination comprising at least one of the foregoing polymers. It is generally desirable for the polymer used in the coating to have a glass transition temperature of less than or equal to about 0° C. Suitable polymers that may be used in the coating are polyolefins, polytetrafluoroethylene, polysiloxanes, polyesters, or the like, or combinations comprising at least one of the foregoing polymers.

When the clamp 36 is made from a shape memory alloy, the clamp 36 may activated by an external stimulus to either promote an expansion or contraction of the clamp 36. The external stimulus is heat supplied by an actuator means, which is also contained in the housing. For shape memory alloys, the external stimulus is an electrically resistive heating of the shape memory alloys either directly or indirectly.

In one embodiment, the clamp 36 may be made from a polymer. If desirable, both the clamp 36 as well as the barrier membrane 22 may be made from a polymer. In another embodiment, the clamp 36 and/or the barrier membrane 22 may be made from a shape memory polymer. Shape memory polymers generally refer to a group of polymeric materials that demonstrate the ability to return to some previously defined shape when subjected to an appropriate thermal stimulus. Generally, shape memory polymers have two main segments, a hard segment and a soft segment. The previously defined or permanent shape can be set by melting or processing the polymer at a temperature higher than the highest thermal transition followed by cooling below that thermal transition temperature. The highest thermal transition is usually the glass transition temperature (T_{g}) or melting point of the hard segment. A temporary shape can be set by heating the material to a temperature higher than the T_{g} or the transition temperature of the soft segment, but lower than the T_{g} or melting point of the hard segment. The temporary shape is set while processing the material at the transition temperature of the soft segment followed by cooling to fix the shape. The material can be reverted back to the permanent shape by heating the material above the transition temperature of the soft segment.

Generally, shape memory polymers are co-polymers comprised of at least two different units which may be described as defining different segments within the co-polymer, each segment contributing differently to the flexural modulus properties and thermal transition temperatures of the material. The term “segment” refers to a block, graft, or sequence of the same or similar monomer or oligomer units that are copolymerized with a different segment to form a continuous, crosslinked, interpenetrating network of these segments. These segments may be combination of crystalline or amorphous materials and therefore may be generally classified as a hard segment(s) or a soft segment(s), wherein the hard segment generally has a higher glass transition temperature (T_{g}) or melting point than the soft segment. Each segment then contributes to the overall flexural modulus properties of the shape memory polymer and the thermal transitions thereof. When multiple segments are used, multiple thermal transition temperatures may be observed, wherein the thermal transition temperatures of the copolymer may be approximated as weighted averages of the thermal transition temperatures of its comprising segments. The previously defined or permanent shape of the shape memory polymer can be set by blow molding the polymer at a temperature higher than the highest thermal transition temperature for the shape memory polymer or its melting point, followed by cooling below that thermal transition temperature.

Suitable examples of thermoplastic shape memory polymers that may be used in the clamp 36 are polyacetals, polyurethanes, polyolefins, polyacrylics, polycarbonates, polystyrenes, polyesters, polyamides, polyamideimides, polyarylates, polyarylsulfones, polyethersulfones, polyphenylene sulfides, polysulfones, polyimides, polyetherimides, polytetrafluoroethylenes, polyetherketones, polyether etherketones, polyether ketone ketones, polybenzoxazoles, polyoxadiazoles, polybenzothiazinophenothiazines, polybenzothiazoles, polypyrazinoquinoxalines, polypyromellitimides, polyquinoxalines, polybenzimidazoles, polyoxindoles, polyoxoisoindolines, polydioxoisoindolines, polytriazines, polypyridazines, polypiperazines, polypyridines, polypiperidines, polytriazoles, polypyrazoles, polycarboranes, polyoxabicyclononanes, polydibenzofurans, polyphthalides, polyacetals, polyanhydrides, polyvinyl ethers, polyvinyl thioethers, polyvinylalcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, or the like, or combinations comprising at least one of the foregoing thermoplastic polymers.

The collection device comprising the clamp 36 and the barrier membrane 22 may be introduced into the patient by means of a deployment device 100 shown in the FIG. 16. FIG. 16 is a depiction of one exemplary embodiment of the lateral external view of the deployment device 100 comprising a housing 10 having a deployment end 12 which is inserted into the patient and which houses the expandable barrier membrane (not shown) in a constrained configuration; a shaft portion 14 which may be partially or completely inserted within the patient body; and an actuator end 16 opposite the deployment end, which is retained substantially outside the patient. The housing 10 can be flexible or rigid, and its rigidity can vary along its length. A remote actuator means 18 is used to project and/or retract, and, optionally, to rotate the barrier membrane relative to the distal deployment opening 24.

FIGS. 17, 18, 19, and 20 show the use of the device to obtain a tissue sample. FIGS. 17, 18, 19, and 20 represent a simplified cross sectional view of the device shown in FIG. 16, the cross section being taken along line a-a of FIG. 16. In use, the device is partially inserted into a human or animal patient (not shown). The housing can be inserted directly into a patient, or the device can be emplaced using an instrument channel of a standard endoscope, laparoscope, catheter, or the like.

FIG. 17 depicts a cross-section of the device of FIG. 16 with the expandable barrier membrane 22 in a first, constrained configuration. The housing 10 is preferably an elongated sheath, having an axial bore 20 therethrough, the axial bore being sized to receive the expandable barrier membrane 22 in a constrained configuration. The axial bore 20 opens to the environment at the deployment opening 24. In one embodiment (not shown), the axial bore also opens to the environment at the actuator opening 26, and access for additional laparoscopic or endoscopic devices, and/or fluid access or withdrawal, is provided. A seal (not shown) may be added at the actuator opening 26, to minimize or prevent fluid (i.e., liquid or gas) leakage.

The specific configuration and dimensions of the axial bore 20 will vary with the use of the device, the parameters of the barrier membrane 22, and whether access for additional laparoscopic or endoscopic devices is provided. In general, the axial bore 20 will have an internal diameter of about 0.1 centimeter (cm) to about 3 cm. In one embodiment, the axial bore 20 has an internal diameter of about 0.25 cm to about 2.5 centimeter. In another embodiment, the axial bore has an internal diameter of about 0.3 cm to about 2 cm. An exemplary internal diameter for the axial bore is 0.9 cm. In one embodiment (not shown), the axial bore comprises a working channel of an endoscope. Such an endoscope can also provide surgical implements such as lasers, scalpels, irrigation and aspiration means, visualization means, and the like.

The outer diameter of the housing 10 will vary with the application, the size of the expandable barrier, and whether additional working channels are included in the device. In general, it is desirable for the housing of a laparoscopic device to have an outer diameter of less than about 1 millimeter (mm) to about 5 cm. In another embodiment, the housing of a laparoscopic device can have an outer diameter of about 2 millimeter (mm) to about 4 cm. In yet another embodiment, the housing of a laparoscopic device can have an outer diameter of about 3 millimeter (mm) to about 3 centimeter (cm). An exemplary housing outer diameter is about 1 cm.

The length of laparoscopic devices can vary in an amount of about 10 cm to about 150 cm. In one embodiment, the length of the laparoscopic device can vary in an amount of about 20 to about 80 cm. In another embodiment, the length can vary in an amount of about 30 to about 70 cm. In yet another embodiment, the length can vary in an amount of about 40 to about 60 cm. An exemplary length of a laparoscopic or endoscopic device is about 25 cm.

The barrier membrane 22 is extended through the deployment opening 24 remotely. The barrier membrane 22 is in mechanical communication with a clamp 36 through the actuator opening 26 of the housing 10 by a connecting means 28. The connecting means 28 can be, for example, affixed to the barrier membrane 22, by a variety of means such as a screw, soldering, the use of an adhesive, or the like. Alternatively, the connecting means 28 can be a continuation of the material used in forming the clamp 36. The clamp 36 is in physical contact with the barrier membrane 22. In the shown configuration, the barrier membrane 22 is attached to the remote actuator means 18 by the connecting means 28. Longitudinal axial movement of the actuator means 18 relative to the housing 10 causes the barrier membrane 22 to be extended from, or retracted into, the housing 10, via the deployment opening 24. Rotational movement of the actuator means 18 relative to the housing 10 causes the barrier membrane 22 to be rotated. If rotational movement is not desirable, a means to prevent rotation can be employed.

In the depicted configurations, the remote actuator means 18 slidably engages the actuator opening 26. The remote actuator means 18 can be an extension of the clamp 36, or of the connecting means 28, and be substantially independent of the housing 10. Alternatively, the remote actuator means 18 can be connected to the connecting means 28 by threads, welding, soldering, or the like.

The housing 10 includes, or provides integration with, a surgical handling apparatus to deploy and retract the barrier membrane. In one embodiment, as shown, two finger rings 30 are part of the actuator end 16. An additional thumb ring 32 is part of the remote actuator means 18. These rings are for ease of handling. Knobs or ridges, for example, can be provided for ease of integration with a separate actuator means (not shown). Suitable actuator means include slider mechanisms, pistol grip or thumb actuated mechanisms, scissors handles, and syringe-plunger mechanisms. These and others are well known to the art. The specific type of actuator mechanism is generally determined by the personal preference of the surgeon.

In use, the deployment end 12, and possibly the shaft portion 14, is inserted into the patient. The housing can be inserted directly into the patient, or it can be introduced using the instrument channel of a standard least invasive surgery (LIS) device. The deployment end 12 possesses lateral integrity such that it is not significantly deformed by the pressure exerted by the constrained barrier membrane 22. In a device having a rigid housing (the usual case for a laparoscopic device), the deployment end 12 of the housing can be integral to the shaft portion 14 of the housing, such that there is no obvious demarcation between the functional zones. When the device functions as a catheter (e.g., endoscopic use) and there is little lateral support, the deployment end 12 may be reinforced to provide consistent constraint of the expandable barrier membrane.

The shaft portion 14 of the housing is located between the actuator (non-inserted) end 16 and the deployment (inserted) end 12 of the device. The shaft portion 14 of the housing may be inserted into the patient (not shown) partially or completely. The shaft portion 14 of a device, which is used in laparoscopy must have sufficient structural integrity that it is easily inserted through a surgical opening into the body of the patient without undue deformation. The material should also not react with the fluids or tissues in the human or animal body. Materials with sufficient structural rigidity include stainless steel and polymeric materials.

The material of the shaft portion 14, and the material of the deployment end 12, can be the same, or can have different physical properties. For example, the shaft portion 14 of an expandable barrier device housing used in endoscopic surgery will generally be flexible, to allow insertion through naturally occurring orifices, ducts, and/or passages, or to allow insertion through the working channel of an endoscope. Suitable examples of polymeric materials include polytetrafluorethylene, polyurethane, polyethylene, or the like. The material of such a flexible housing may be reinforced at the deployment end 12 with fibers, rings, or longitudinal ribs, for example, to enable it to withstand the forces exerted on it by the barrier membrane 22 while it is constrained within and deformed by the housing.

When expanded, the barrier membrane 22 can have a diameter of from about 1 cm or less to about 10 cm or greater, more generally from about 2 cm to about 6 cm. The barrier membrane 22 spans the clamp 36 loosely, forming a rounded plate or bowl. The depth of arc described by the barrier membrane 22 when suspended from the clamp 36 is from less than about 1 cm to about 7 cm or greater. In general, the preferred depth of the pouch formed by the barrier membrane 22 will be less when the barrier membrane 22 is used primarily as a tissue protecting surgical drape, and will be correspondingly greater when the barrier membrane is used as a pouch to collect tissue or to remove tissue in situ from the surgery site. In those embodiments in which a relatively deep bowl-like pouch is present, it may be desirable to reinforce the barrier membrane. Reinforcing stays, filaments or ribs, made of, for example, plastic, thickened barrier membrane material, or a shape memory alloy, provide reinforcement, and assist the barrier membrane to deploy fully into the desired shape.

The barrier membrane 22 is compressed and loaded within the axial bore 20. In this constrained configuration, the barrier device can be sterilized, packaged and stored for later use. Preferably at least one expandable barrier device is available during surgery: when needed, the surgeon can visually assess the size of the barrier membrane necessary for tissue protection and/or collection, and select an appropriate expandable barrier device. When constrained, the barrier membrane 22 is collapsed, and may be furled around the clamp 36.

FIG. 18 shows the collection device of FIG. 17 in an expanded position. The remote actuator means 18 has been moved distally along the axial bore 20. The clamp 36 extends past the confines of the deployment opening 24. Once the clamp 36 is released from the compression of the housing 10, the loop regains its unconstrained, memory, shape and the barrier membrane 22 attains its deployed configuration. While the clamp 36 is shown as generally circular or oval, as noted above, other shapes are also possible. Elliptical, rounded, square, and irregular shapes are also possible, and may be desirable for a particular application.

The barrier membrane 22 is connected to the clamp 36. As the clamp 36 expands, the barrier membrane 22 unfurls to form a generally plate-like or bowl-like enclosure having a mouth 38. The perimeter, or the mouth 38, of the barrier membrane 22 is defined by the intersection of the clamp 36 and the barrier membrane 22. The more bowl-like configuration, shown in FIG. 18, is generally preferred when the device is used to collect or retrieve tissue samples. In use, the expanded barrier membrane 22 is suspended internally at or near the surgical site. The barrier can be manipulated to underlie the surgical site, so that fluids or other materials that are released at the surgical site flow gently downhill into the expandable barrier by means of irrigation flow and/or gravity. When the barrier membrane 22 is bowl-like, it can substantially contain a tissue sample 40 to be excised and removed during surgery.

FIG. 19 shows the device of FIG. 18 in a pouched configuration, partially between the expanded configuration of FIG. 18 and the withdrawal configuration of FIG. 20. The remote actuator means 18 has been moved proximally along the inside of the axial bore 20. The clamp 36 extends only partially past the confines of the deployment opening 24, and constraining force of the housing 10 has forced the clamp 36 into a deformed, semi-constrained shape. The barrier membrane 22 can preferably slide relative to the clamp 36. The barrier membrane 22 is preferably not retracted into the housing 10 with the clamp 36, and remains substantially outside of the housing 10. As the clamp 36 is withdrawn into the housing 10, the barrier membrane 22 catches on the deployment opening 24 of the deployment end 12 of the housing 10. Therefore, the diameter of the mouth 38 of the barrier membrane 22 becomes reduced as compared to the expanded configuration shown in FIG. 18, and the barrier membrane 22 forms a pouch. The tissue sample 40 is substantially enclosed in the pouch.

FIG. 20 shows the device of FIG. 19 in a configuration for withdrawal from the body. The remote actuator means 18 has been moved further along the axial bore 20 in the proximal direction, and is in approximately the position from which it started. The clamp 36 is substantially fully retracted into the axial bore 20, and constraint of the housing 10 has deformed the clamp 36 to fit within the axial bore 20. The mouth 38 of the barrier membrane 22 is retracted into the housing 10 with the clamp 36, preventing any undesired loss of tissue or fluids from within the pouch. The body of the barrier membrane 22, containing the tissue sample 40, remains substantially outside of the housing 10. In this configuration the device is withdrawn. As the filled pouch of the barrier membrane 22 is generally larger than the deployment opening 24, there is a tendency for the barrier membrane 22 to seal against the deployment opening 24 of the housing 10. This tendency can be enhanced by placing a seal or gasket means (not shown) at the deployment opening 24.

While the demonstration of the deployment device 100 and the collection device as shown in the FIG. 16 through FIG. 20 is representative of one embodiment of a device of this invention, other embodiments are also within the scope of the invention. For example, in an alternate embodiment, not shown, the barrier membrane 22 is adhered to the clamp 36, so that as the mouth of the barrier membrane 22 is withdrawn into the housing 10 it is only collapsed transversely as the clamp 36 is withdrawn into and contained within the axial bore. In yet another embodiment, the barrier membrane and tissue sample are completely withdrawn into the housing for removal from the body.

The pouched barrier membrane 22 can provide a transfer means for tissues that have been removed from a patient and are to be delivered, for example, to a pathology laboratory. The entire barrier device can be delivered, or the distal end of the device including the pouched barrier membrane can be separated from the rest of the device and delivered (not shown). If such a transfer is desired, the barrier membrane can be lined with, can contain, or can be filled with a tissue preservative.

The devices of this invention, including the housing and the barrier membrane, are reusable. Preferably the device is disposable or semidisposable. The barrier membrane and the housing are generally disposable, and the remote actuator means is either reused or discarded.

In one embodiment, in one method of using the device, the housing is inserted into the body of a living being following which the actuating means located in the proximal end of the housing is used to actuate the clamp. The longitudinal motion of the clamp permits it to be extended (ejected) from the housing. When the clamp contains an inflexion point or when the clamp is manufactured from two elements that are not in mechanical communication with each other, its ejection from the housing promotes it to expand as a result of its spring like characteristics. When the clamp is manufactured from a conduit, the conduit may be expanded by the use of compressed air. The clamp and the barrier membrane generally expand upon ejection from the housing. Since the clamp is in mechanical communication with the barrier membrane, the expansion of the clamp facilitates an opening of the mouth of the barrier membrane. The clamp and the barrier membrane may then be utilized to retrieve matter from within the body. Following the retrieval of matter from within the body, the clamp together with the barrier membrane may be withdrawn into the housing. Alternatively, the barrier membrane may be left behind inside the body to be retrieved later. In another embodiment, after a first retrieval, the clamp and the barrier membrane may be deployed and manipulated to remove additional matter from another part of the body before being withdrawn into the housing.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A collection device for manipulating matter in an inaccessible space, comprising
a clamp in mechanical communication with an actuator means, wherein the clamp has an inflexion point;
a barrier membrane in mechanical communication with the clamp, wherein the expansion of the barrier membrane is controlled by the clamp.

2. The collection device of claim 1, wherein the contraction of the barrier membrane is controlled by the clamp.

3. The collection device of claim 1, wherein the clamp comprises a metal, a polymer, or a metal and a polymer.

4. The collection device of claim 4, wherein the metal is a shape memory alloy and wherein the polymer is a shape memory polymer.

5. The collection device of claim 1, wherein the barrier membrane comprises a polymer and wherein the barrier membrane is disposable or replaceable.

6. The collection device of claim 1, wherein the inflexion point is located on a longitudinal axis of the clamp; wherein the longitudinal axis of the clamp is an axis that can divide the clamp into equal parts.

7. The collection device of claim 1, wherein the inflexion point is located on a longitudinal axis of the clamp; wherein the longitudinal axis of the clamp is an axis that can divide the clamp into two equal halves.

8. The collection device of claim 7, wherein the inflexion point is located on a longitudinal axis of the clamp, wherein the longitudinal axis of the clamp is superimposed upon a longitudinal axis of a tubular housing.

9. The collection device of claim 1, wherein the inflexion point is the point of maximum deformation upon the application of a force to the clamp.

10. The collection device of claim 1, wherein the inflexion point is caused by a reduction in cross-sectional area of the clamp or wherein the inflexion point is caused by a reduction in elastic modulus of the material of the clamp.

11. The collection device of claim 10, wherein the reduction in the cross-sectional area is attained by incorporating a notch or a hole in the clamp.

12. The collection device of claim 10, wherein the reduction in elastic modulus is attained by incorporating a material having a different composition from that of the remainder of the clamp into the clamp.

13. The collection device of claim 1, wherein the inflexion point is caused by a predeformed section that is created by bending a section of the clamp in a manner such that the bend includes an internal angle of less than or equal to about 180 degrees as measured by the angle between two tangents taken to the bend when these tangents meet on the longitudinal axis that divides the clamp into two equal halves when the clamp is in its undeformed state, and wherein the tangents to the bend are taken at two points of maximum curvature, one on either side of the longitudinal axis.

14. The collection device of claim 1, wherein the inflexion point is caused by a predeformed section that is created by bending a section of the clamp in a manner such that the bend includes an internal angle of less than or equal to about 90 degrees as measured by the angle between two tangents to the bend when these tangents meet on the longitudinal axis that divides the clamp into two equal halves when the clamp is in its undeformed state, and wherein the tangents to the bend are taken at two points of maximum curvature, one on either side of the longitudinal axis.

15. The collection device of claim 1, wherein the inflexion point is caused by a predeformed section having a U-shape, a V-shape, a W-shape, a M-shape, a S-shape, a O-shape, or a combination comprising at least one of the foregoing shapes.

16. The collection device of claim 1, wherein the inflexion point is not located on a longitudinal axis of the clamp or the housing.

17. The collection device of claim 1, wherein the clamp has more than one inflexion point, and wherein the inflexion points are not located on a longitudinal axis of the clamp or the housing.

18. The collection device of claim 1, wherein the expansion of the clamp leads to the formation of a mouth in the barrier membrane.

19. The collection device of claim 18, wherein the mouth is not formed in a single plane.

20. The collection device of claim 18, wherein the mouth is not formed in a plane that contains a longitudinal axis of a housing or the clamp.

21. The collection device of claim 1, wherein the clamp is manufactured from a conduit.

22. The collection device of claim 1, wherein the clamp is manufactured from a conduit and wherein the clamp can expand and contract upon the application and removal of hydraulic or pneumatic pressure.

23. The collection device of claim 21, wherein the clamp and a barrier membrane are manufactured from the same material.

24. The collection device of claim 22, wherein the clamp and the barrier membrane are manufactured from an elastomer.

25. The collection device of claim 22, wherein the clamp and the barrier membrane are manufactured from a polymer.

26. The collection device of claim 25, wherein the clamp and the barrier membrane are manufactured from polyvinyl chloride, polysiloxanes, polyurethane, fluoropolymers, polyesters, polyamides, polyolefins, or a combination comprising at least one of the foregoing polymers.

27. The collection device of claim 21, wherein the clamp is in the form of a web or a grid.

28. The collection device of claim 1, wherein the clamp is in mechanical communication with the barrier membrane via a hook, threads, a magnet, an adhesive, a spring, or a combination comprising at least one of the foregoing.

29. The collection device of claim 1, wherein the clamp and/or the barrier membrane comprises a polymer, wherein the polymers are polyacetals, polyurethanes, polyolefins, polyacrylics, polycarbonates, polystyrenes, polyesters, polyamides, polyamideimides, polyarylates, polyarylsulfones, polyethersulfones, polyphenylene sulfides, polysulfones, polyimides, polyetherimides, polytetrafluoroethylenes, polyetherketones, polyether etherketones, polyether ketone ketones, polybenzoxazoles, polyoxadiazoles, polybenzothiazinophenothiazines, polybenzothiazoles, polypyrazinoquinoxalines, polypyromellitimides, polyquinoxalines, polybenzimidazoles, polyoxindoles, polyoxoisoindolines, polydioxoisoindolines, polytriazines, polypyridazines, polypiperazines, polypyridines, polypiperidines, polytriazoles, polypyrazoles, polycarboranes, polyoxabicyclononanes, polydibenzofurans, polyphthalides, polyacetals, polyanhydrides, polyvinyl ethers, polyvinyl thioethers, polyvinylalcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, polysiloxanes, or combinations comprising at least one of the foregoing thermoplastic polymers.

30. The collection device of claim 1, wherein the clamp and/or the barrier membrane comprises a shape memory alloy or a shape memory polymer, wherein the shape memory alloy comprises nickel and titanium.

31. The collection device of claim 1, wherein the clamp and/or the barrier membrane comprises a β titanium alloy having molybdenum equivalent of about 7 to about 11 wt %, based upon the total weight of the alloy, wherein the Mo equivalent in weight percent is calculated according to the following equation (2):
MO_{eq.}=1.00Mo+0.28Nb+0.22Ta+0.67V+1.43Co+1.60Cr+0.77Cu+2.90Fe+1.54Mn+1.11Ni+0.44W+0.25(Sn+Zr+Hf)−1.00Al (2)
wherein Mo is molybdenum, Nb is niobium, Ta is tantalum, V is vanadium, Co is cobalt, Cr is chromium, Cu is copper, Fe is iron, Mn is manganese, Ni is nickel, W is tungsten, Al is aluminum, Sn is tin, Zr is zirconium, Hf is hafnium, and wherein the respective chemical symbols represent the amounts of the respective elements in weight percent based on the total weight of the alloy.

32. A collection device for manipulating matter in an inaccessible space, comprising
a clamp in mechanical communication with an actuator means, wherein the clamp comprises at least two elements that can control a barrier membrane and wherein the at least two elements are not in mechanical communication with one another;
a barrier membrane in mechanical communication with the at least two elements of the clamp, wherein the expansion of the barrier membrane is controlled by the clamp.

33. The collection device of claim 32, wherein a contraction of the barrier membrane is controlled by the clamp.

34. The collection device of claim 32, wherein the clamp comprises a metal, a polymer, or a metal and a polymer.

35. The collection device of claim 34, wherein the metal is a shape memory alloy and wherein the polymer is a shape memory polymer.

36. The collection device of claim 32, wherein the barrier membrane comprises a polymer.

37. A surgical apparatus for manipulating matter at an intended manipulation temperature in a confined or inaccessible space, comprising:
a housing;
a clamp in located at the distal end of the housing in slideable communication with the housing, wherein the clamp has an inflexion point;
a barrier member in communication with the clamp; and
an actuating means located at the proximal end of the housing, for extending the clamp from the housing to manipulate matter within the space and for withdrawing the clamp into the housing, the arrangement being such that the clamp bends or twists in a lateral or helical sense to manipulate the matter on extending from the housing at the manipulation temperature, and wherein the clamp becomes relatively straightened on withdrawal into the housing at the manipulating temperature.

38. The apparatus of claim 37, which is of elongate form for surgical manipulation of matter within the interior of a living body, and which has the clamp located at its distal end with the actuating means being operable from the proximal end of the apparatus.

39. The apparatus of claim 37, wherein the clamp comprises a shape memory alloy that is pseudoelastic or superelastic at the manipulation temperature.

40. The apparatus of claim 37, wherein the clamp comprises a shape memory polymer.

41. The apparatus of claim 37, wherein the barrier membrane is disposable and wherein the barrier membrane is manufactured from a polymer.

42. The apparatus of claim 37, further comprising a drawstring that is used to close a mouth of the barrier membrane, and wherein the drawstring is manipulated at the proximal end of the apparatus.

43. The apparatus of claim 37, wherein the barrier membrane can be detached from the clamp inside the body cavity and left behind for later removal.

44. A method for manipulating matter within a confined space or an inaccessible space inside a living being comprising:
inserting into the body of a living being a surgical apparatus comprising:
a housing;
a clamp in located at the distal end of the housing in slideable communication with the housing, wherein the clamp has an inflexion point;
a barrier member in communication with the clamp; and
an actuating means located at the proximal end of the housing and in mechanical communication with the clamp;
extending the clamp from the housing to manipulate matter within the space; and
withdrawing the clamp into the housing.

45. The method of claim 44, wherein the clamp bends or twists in a lateral or helical sense to manipulate the matter on extending from the housing at a manipulation temperature, and wherein the clamp becomes relatively straightened on withdrawal into the housing at the manipulating temperature.

46. A method for manipulating matter within a confined space or an inaccessible space inside a living being comprising:
inserting into the body of a living being a surgical apparatus comprising:
a housing;
a barrier member in communication with the clamp;
a clamp in mechanical communication with an actuator means, wherein the clamp comprises at least two elements that can control a barrier membrane and wherein the at least two elements are not in mechanical communication with one another; and further wherein the clamp is located at the distal end of the housing in slideable communication with the housing; and
an actuating means located at the proximal end of the housing;
extending the clamp from the housing to manipulate matter within the space; and
withdrawing the clamp into the housing.
